(19) United States
(12) Patent Application Publication
(10) Pub. No.: US 2022/0080114 A1
(43) Pub. Date: Mar. 17, 2022

(54) DEVICE, METHOD, AND TUBE DEVICE FOR DELIVERY OF INFUSATE FOR HUMANS

(71) Applicant: THE WAVE TALK, INC., Daejeon (KR)

(72) Inventor: Young Dug KIM, Gyeonggi-do (KR)

(21) Appl. No.: 17/425,514

(22) PCT Filed: Jan. 14, 2020
(86) PCT No.: PCT/KR2020/000668

§ 371 Date: Jul. 23, 2021

(30) Foreign Application Priority Data
Jan. 25, 2019 (KR) 10-2019-0009807
Jan. 25, 2019 (KR) 10-2019-0009808

(51) Int. Cl. A61M 5/142; A61M 5/168

(52) U.S. Cl. CPC A61M 5/14276; A61M 5/16881; A61M 5/16831; A61M 2205/3344; A61M 2205/27; A61M 2205/3303; A61M 2205/3306

(57) ABSTRACT

The present invention relates to a device, a method, and a tube device for delivery of an infusate for humans, which can prevent the infusate from being contaminated by bacteria or microorganisms. The device may comprise: a body; a pump unit installed in the body and forming a pressure difference inside the tube device so as to deliver an infusate for humans; and an infusate contamination sensing unit installed in the body and capable of sensing contamination of the infusate for humans by bacteria or microorganisms in a non-contact manner by using the optical characteristics of the infusate for humans.

### TECHNICAL FIELD

The present invention relates to a device, method, and tube device for delivery of an infusate for humans, and more particularly, to a device, method, and tube device for delivery of an infusate for humans which can prevent the infusate from being contaminated by bacteria or microorganisms.

### BACKGROUND ART

A device capable of automatically injecting an infusate may be used for administration of infusate for humans, such as Ringer's solution, an analgesic agent, or an anticancer agent, which needs to be continuously administered in small amounts for a certain period of time depending on a patient.

There may be two broad types of devices capable of automatically delivering an infusate for humans: so-called, a syringe pump and an infusion pump.

In general, a syringe pump, which is a device that is equipped with a syringe filled with an infusate for humans and mechanically presses a plunger of the syringe, can continuously inject one dose of the infusate for a predetermined period of time, and an additional injection may be administered by replacing the syringe if necessary.

This may not be suitable for patients who require continuous administration of a large volume of infusate because the capacity of a syringe is relatively small and the infusate administration is interrupted during syringe replacement.

In addition, the syringe pump has to be fixed in a place close to a patient, and accordingly brings inconvenience to the patient who is mobile.

On the contrary, a conventional infusion pump is suitable for administering a large amount of infusate for humans over a prolonged time period because it can continuously administer the infusate supplied from a pouch or bottle in which the infusate is stored, and especially, the infusion pump may provide convenience for a patient to carry it and move.

On the other hand, infusate for humans, such as Ringer's solution, an analgesic agent, or an anticancer agent, which is to be infused into the human body or living bodies of animals and plants may be contaminated by bacteria or microorganisms during the manufacturing process, storage process, injection process, or dispensing process thereof.

Various contamination accidents occurring in the medical field by such contaminated infusate cause fatal loss of life, and there were frequent cases where the contaminated infusate was injected into the elderly or dispensed into several people, resulting in fatal and large-scale medical incidents.

### Technical Problem

An object of the present invention is to provide a device, method, and tube device for delivery of an infusate for humans which enable the prevention of a medical incident by making it possible to determine in real-time or periodically contamination of an infusate for humans by bacteria or microorganisms before infusion into the human body or the living body of an animal or plant. However, the above object is illustrative only, and does not limit the scope of the present invention.

### Technical Solution

According to an aspect of the present invention to achieve the above object, there is provided a device for delivery of an infusate for humans including: a body; a pump unit installed in the body and forming a pressure difference inside the tube device so as to deliver an infusate for humans; and an infusate contamination sensing unit installed in the body and capable of sensing contamination of the infusate for humans by bacteria or microorganisms in a non-contact manner by using the optical characteristics of the infusate for humans.

In addition, according to the present invention, the infusate contamination sensing unit may be a non-contact speckle sensor capable of emitting laser light to the infusate for humans and detecting a speckle pattern generated due to multiple scattering for the infusate.

In addition, according to the present invention, the infusate contamination sensing unit may include a frame formed in a shape surrounding at least a portion of the tube device; a laser source unit formed on one side of the frame and configured to emit the laser light to the tube device through a light incident portion formed in the frame; and a camera unit formed on the other side of the frame and capable of photographing a multiple-scattering speckle pattern received through a light exit portion formed in the frame.

In addition, according to the present invention, the frame may have an inner diameter surface formed therein corresponding to the tube device, and a light scattering surface or a reflective surface for scattering the laser light may be formed on the inner diameter surface.

In addition, according to the present invention, the frame may include a first frame formed in a case of the body and having a first inner diameter surface formed therein corresponding to one side surface of the tube device is formed; and a second frame having a second inner diameter surface formed therein corresponding to the other surface of the tube device and formed on a cover covering the case of the body so as to be fastened to the first frame.

In addition, according to the present invention, alignment grooves into which ring-shaped alignment protrusions of the tube device are inserted may be formed on the first inner surface and the second inner surface.

In addition, according to the present invention, the frame may further include a fastening device configured to fasten the first frame and the second frame.

Also, according to the present invention, the fastening device may be a magnetic object installed in the first frame or the second frame.

In addition, according to the present invention, a main exit optical axis or the light incident portion of the laser source unit may be formed to be inclined at a first angle toward the camera unit with respect to a vertical direction of the frame.

Also, according to the present invention, the light incident portion and the light exit portion may be formed to be spaced apart by a first distance in a longitudinal direction of the frame.

In addition, the device for delivery of an infusate for humans according to the present invention may further include an infusate flow blocking device formed in the body and configured to operate a knob of a valve installed in the tube device or pressurize the tube device in order to block a flow of the infusate inside the tube device.

Also, according to the present invention, the infusate flow blocking device may include a first valve knob-turning device configured to turn a first knob of a first valve installed at a front end of the optical portion of the tube device; and a second valve knob-turning device configured to turn a second knob of a second valve installed at a rear end of the optical portion of the tube device.

Further, the device for delivery of an infusate for humans according to the present invention may further include a pressure regulating device formed in the body and configured to regulate a pressure or flow rate inside the tube device.

In addition, according to the present invention, the tube device may include a non-optical portion made of a flexible material to correspond to the pump unit and an optical portion made of a light-transmitting material to correspond to the infusate contamination sensing unit.

According to an aspect of the present invention to achieve the above object, there is provided a method of injecting an infusate for humans, which uses a device for delivery of an infusate for humans that includes a body, a pump unit installed in the body and forming a pressure difference inside the tube device so as to deliver an infusate for humans, and an infusate contamination sensing unit installed in the body and capable of sensing contamination of the infusate for humans by bacteria or microorganisms in a non-contact manner by using the optical characteristics of the infusate for humans, the method including: delivering the infusate for humans using the pump unit; blocking or controlling a flow of the infusate for humans by using a valve; detecting the optical characteristics of the infusate for humans by using the infusate contamination sensing unit; determining contamination of the infusate for humans by bacteria or microorganisms by using the optical characteristics; and when it is determined that the infusate is contaminated by bacteria or microorganisms, stopping delivery of the infusate for humans and warning of the same.

On the other hand, according to an aspect of the present invention to achieve the above object, there is provided a tube device that is applicable to a device for delivery of an infusate for humans including a body, a pump unit installed in the body and forming a pressure difference inside the tube device so as to deliver an infusate for humans, and an infusate contamination sensing unit installed in the body and capable of sensing contamination of the infusate for humans by bacteria or microorganisms in a non-contact manner by using the optical characteristics of the infusate for humans, the tube device including a non-optical portion made of a flexible material to correspond to the pump unit; an optical portion made of a light-transmitting material to correspond to the infusate contamination sensing unit; and a valve connected to the non-optical portion at one end thereof, connected to the optical portion at the other end thereof, and capable of blocking a flow of the infusate for humans.

In addition, according to the present invention, the valve may include a first valve formed at a front end of the optical portion and a second valve formed at a rear end of the optical portion.

### Advantageous Effects

According to an embodiment of the present invention as described above, it is possible to prevent in advance medical incidents by making it possible to determine in real-time or periodically determination contamination of an infusate for humans by bacteria or microorganisms before the infusate is injected into the human body or the living body of an animal or plant. However, this effect does not limit the scope of the present invention.

### DESCRIPTION OF DRAWINGS

FIG. 1 is a conceptual diagram illustrating a device for delivery of an infusate for humans according to some embodiments of the present invention.

FIG. 2 is an exploded perspective view showing parts of a device for delivery of an infusate for humans and a tube device according to some other embodiments of the present invention.

FIG. 3 is a cross-sectional view of assembled parts of the device for delivery of an infusate for humans and the tube device of FIG. 2.

FIG. 4 is a flowchart illustrating a method of injecting an infusate for humans according to some embodiments of the present invention.

### MODE FOR INVENTION

The invention is described more fully hereinafter with references to the accompanying drawings, in which exemplary embodiments of the invention are shown. This invention may, however, be embodied in many different forms and should not be construed as limited to the embodiments set forth herein. Rather, these exemplary embodiments are provided so that this disclosure is thorough, and will fully convey the scope of the invention to those skilled in the art. In addition, the size of each component in the drawings may be exaggerated or reduced for the convenience of explanation.

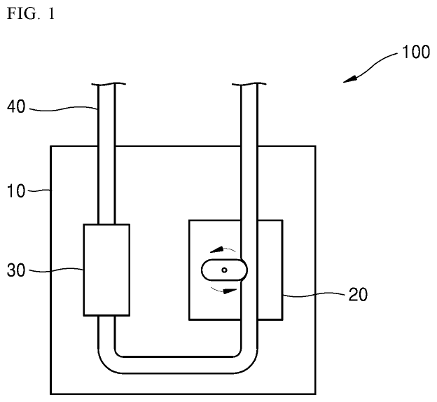

FIG. 1 is a conceptual diagram illustrating a device 100 for delivery of an infusate for humans according to some embodiments of the present invention.

First, as shown in FIG. 1, the device 100 for delivery of an infusate for humans according to some embodiments of the present invention may broadly include a body 10, a pump unit 20, and an infusate contamination sensing unit 30.

Here, for example, the body 10 may be of a structure provided therein with a receiving space to accommodate the pump unit 20 and the infusate contamination sensing unit 30 and having strength and durability sufficient to support these units.

In addition, for example, the pump unit 20, which is installed in the receiving space of the body 10, may be a type of pressure-forming device that forms a pressure difference inside a tube device 40 so that an infusate 1 for humans that can be injected into the human body or the living body of an animal or plant, such as Ringer's solution, an analgesic agent, or an anticancer agent, can be delivered in a direction of the human body or the living body.

Here, although not shown, all types of various pumping devices, such as a syringe device, a rotary device, or a piezoelectric element that can be applied to a conventional syringe pump or infusion pump are applicable to the pump unit 20.

In addition, for example, the infusate contamination sensing unit 30, which is installed in the receiving space of the body 10, may include a type of biosensor capable of detecting contamination by bacteria or microorganisms in a non-contact manner by using optical characteristics of the infusate 1.

More specifically, for example, the infusate contamination sensing unit 30 may be a non-contact speckle sensor capable of emitting laser light L to the infusate 1 and detecting a speckle pattern generated due to multiple scattering for the infusate, and the principle of a chaotic wave sensor may be applied thereto.

For example, according to a principle of a chaotic wave sensor, in the case of a material with homogeneity of internal refractive index, such as glass, refraction occurs in a certain direction when coherent light is irradiated thereto.

However, when coherent light, such as laser light, is irradiated onto an object having a heterogeneous internal refractive index or formed of fine refraction or scattering protrusions, very complex multiple scattering occurs inside the material.

Some of the light rays that have been scattered through complicated paths due to multiple scattering pass through the infusate, which is a test target object. Light rays passing through multiple points in the test target object generate constructive interference or destructive interference, and the constructive/destructive interference of the light rays generates grain patterns (speckles).

The light rays scattered along the complicated paths are referred to as “chaotic waves,” and the chaotic waves may be detected through coherent light speckles, and in the case where the coherent light is laser light, the coherent light speckles may be detected through laser speckles.

When a stable medium is irradiated with coherent light, i.e., when a stable medium, in which an internal component does not move, is irradiated with coherent light (e.g., laser light), a stable speckle pattern without a variation may be observed.

However, when an unstable medium having an internal component that is moving, such as bacteria, is included therein, the speckle pattern changes.

That is, due to microscopic biological activities of microorganisms (e.g., intracellular movement, movement of microorganisms, etc.), an optical path may slightly change over time. Since the speckle pattern is generated by interference of light, a fine change in the optical path may cause variation in the speckle pattern. Accordingly, when a temporal variation in the speckle pattern is measured, the biological activities of microorganisms may be rapidly measured. As such, when the variation in the speckle pattern over time is measured, the presence or absence of microorganisms and concentration thereof may be identified, and furthermore, types of microorganisms may also be identified.

The test target object described in this specification is the infusate 1 inside the tube device 40, and a configuration for measuring the variation in the speckle pattern of the infusate over time may be defined as a chaotic wave sensor.

The chaotic wave sensor of the present invention may be configured in various types, such as a reflective type and a transmissive type, and an optical system may be configured in a packaging type.

In addition, the infusate contamination sensing unit 30 may use laser light L having good coherence as a light source. However, in addition to the laser light source, a light source having improved coherence by including a filter that passes only a wavelength of a specific band or specific wavelength in a general illumination source may be used. Alternatively, the measurement may be performed using a wavelength (e.g., infrared, ultraviolet, etc.) outside the visible light range.

Also, the infusate contamination sensing unit 30 may use a camera that is a photographing device for photographing an image. When the infusate is irradiated with coherent light, a coherent light speckle may be formed by multiple scattering. If viruses, bacteria, microorganisms, etc. are present in the infusate, the presence and absence of bacteria and microorganisms and concentration thereof in the test target object may be rapidly determined based on a pattern of the coherent light speckles that vary over time.

For example, as the test target object is irradiated with coherent light every reference time at regular intervals, a coherent light speckle may be formed in the test target object, and as the test target object in which multiple scattering occurs is photographed using the camera or the like, a coherent light speckle image of the formed coherent light speckle may be generated. In this case, a camera including a two-dimensional image sensor or a one-dimensional optical sensor may be used to measure a speckle pattern of a plurality of generated images. For example, a camera equipped with an imaging device, such as a charge-coupled device (CCD) may be used as a measurement unit.

Therefore, whether bacteria and microorganisms are present in the infusate may be determined from the photographed coherent light speckle images in a non-contact manner by checking whether the coherent light speckles are changed in pattern over time. For example, if there is no activity in the infusate, coherent light speckles appear with a constant interference pattern over time. That is, if there is no activity, a constant interference pattern of the coherent light speckles may be found in the images of the coherent light speckles measured every reference time. As such, when the coherent light speckle images show no, or very little, variation of the interference pattern over time, a control unit, which will be described below, may determine that bacteria and microorganisms are absent in the infusate.

On the other hand, when the pattern of the coherent light speckles varies, the control unit may determine that bacteria and microorganisms are present in the infusate. That is, when bacteria and microorganisms are present in the infusate, the bacteria and microorganisms may multiply over time, and the bacteria and microorganisms may continuously move. This movement of the bacteria or microorganisms may cause the continuous change of the pattern of laser speckles over time. Accordingly, when the pattern of the coherent light speckles has changed by a degree equal to or greater than a predetermined error range in the coherent light speckle images measured every reference time, the control unit may determine that bacteria and microorganisms are present in the infusate.

In this case, the degree of change in the pattern of the coherent light speckles may be determined according to the concentration of bacteria and microorganisms. Accordingly, the concentration of the bacteria and microorganisms may be measured through temporal correlation analysis. For example, a standard deviation of light intensity of coherent light speckles may be used to measure the degree of change in the pattern of the coherent light speckles.

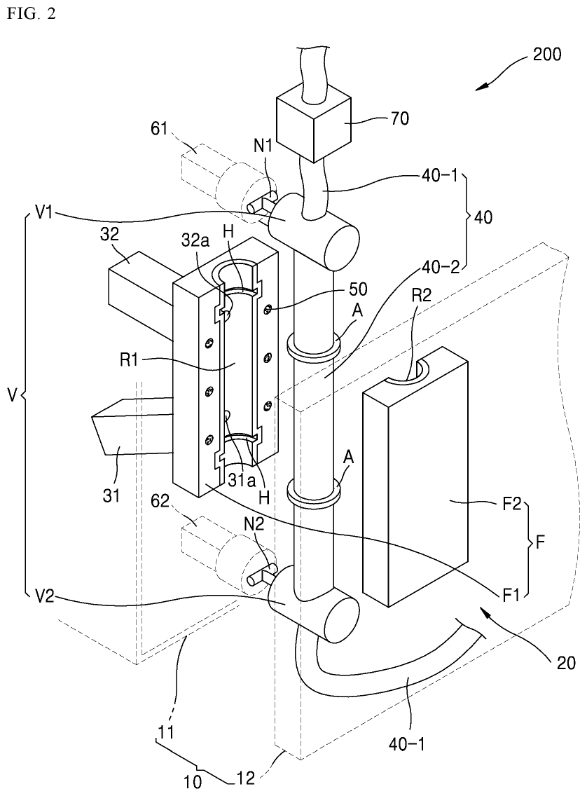

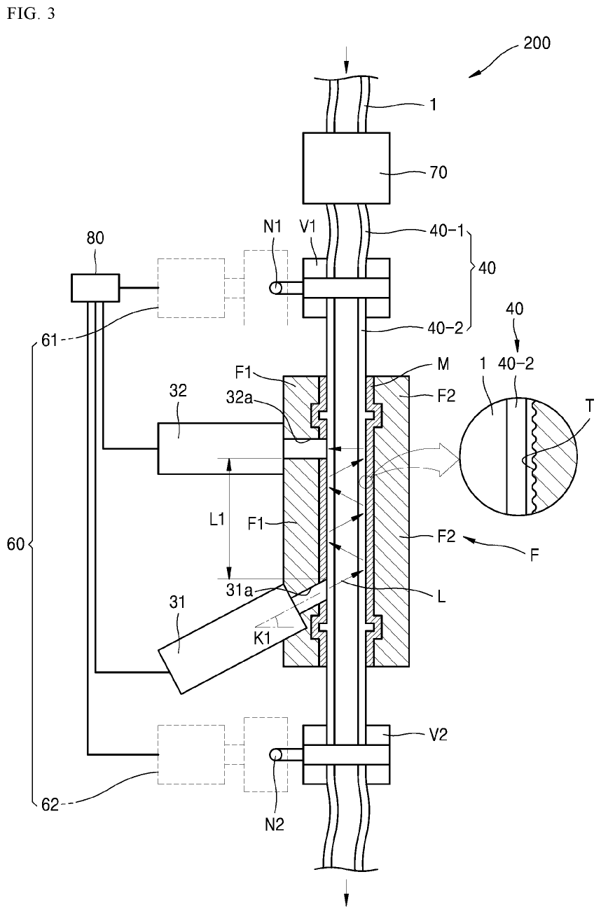

FIG. 2 is an exploded perspective view showing parts of a device 200 for delivery of an infusate for humans and a tube device 40 according to some other embodiments of the present invention, and FIG. 3 is a cross-sectional view of assembled parts of the device 200 for delivery of an infusate for humans and the tube device 40 of FIG. 2.

For example, as shown in FIGS. 2 and 3, an infusate contamination sensing unit 30 of the device 200 for delivery of an infusate for humans according to some other embodiments of the present invention may include a frame F formed in a shape surrounding at least a portion of the tube device 40, i.e., an optical portion 40-2 formed of a light-transmitting material to enable identification of optical characteristics of an infusate 1 for humans, which will be described below, a laser source unit 31 formed on one side of the frame F and configured to emit laser light L to the tube device 40 through a light incident portion 31a formed in the frame F, and a camera unit 32 formed in the other side of the frame F and capable of photographing a speckle pattern of the infusate generated due to multiple scattering which is received through a light exit portion 32a formed in the frame F.

Here, as a spectral bandwidth of a light source that determines the coherence of the laser light L of the laser source unit 31 decreases, the measurement accuracy may increase. That is, as the coherence length increases, the measurement accuracy may increase. Accordingly, a laser light source having a spectral bandwidth of a light source less than a predefined reference bandwidth may be used as the laser source unit 31, and the measurement accuracy may increase as the spectral bandwidth of the light source is shorter than the reference bandwidth. For example, in order to measure the change in the pattern of laser speckles, the spectral bandwidth of the light source may be maintained to be less than 1 nm when light is irradiated into a culture dish every reference time.

On the other hand, for example, as shown in FIGS. 2 and 3, the frame F may have therein an inner diameter surface corresponding to the tube device 40, and a light scattering surface T or a reflective surface for scattering the laser light may be formed on the inner diameter surface.

The frame F has an outer surface formed of a resin or ceramic material, and may be made of a duplex material in which a metal layer M or a mirror layer is formed on the inner diameter surface.

In addition, the frame F may be formed by coating scattering materials that can cause scattering on the inner diameter surface.

However, the present invention is not necessarily limited thereto, and the frame F may be made of a metal material having excellent reflectivity as a whole.

More specifically, for example, as shown in FIGS. 2 and 3, the frame F may be divided into two parts so that the tube device 40 can be easily inserted therein.

That is, as shown in FIGS. 2 and 3, the frame F may include: a first frame F1 formed in a case 11 of the body 10 and having a first inner diameter surface R1 formed thereon corresponding to one side surface of the tube device 40; and a second frame F2 having a second inner diameter surface R2 formed thereon corresponding to the other side surface of the tube device 40 and formed on a cover 12 covering the case 11 of the body 11 so as to be fastened to the first frame F1.

Here, alignment grooves H into which ring-shaped alignment protrusions A of the tube device 40 are inserted may be formed on the first inner diameter surface and the second inner diameter surface for the alignment of the tube device 40.

Thus, by fastening the first frame F1 and the second frame F2 to each other after positioning the tube device 40 between the first frame F1 and the second frame F2, the tube device 40 can be used for one-time use.

At this time, by engaging the alignment protrusions A with the alignment grooves H, the tube device 40 may be aligned at a correct position.

Thereafter, when the tube device 40 is discarded after use, the tube device 40 may be easily removed by separating the first frame F1 and the second frame F2.

In this case, for this purpose, the frame F may further include a fastening device 50 configured to fasten the first frame F1 and the second frame F2.

Here, the fastening device 50 may be, as shown in FIGS. 2 and 3, a magnetic object, such as a permanent magnet, an iron material, or the like, which is installed to correspond to each other in the first frame F1 or the second frame F2.

However, the fastening device 50 is not necessarily limited to the magnetic object, and all types of various fastening devices, such as screws, bolts, nuts, forced-engagement fastening devices, hinges, zippers, buttons, Velcro tapes, snap buttons, etc., are applicable.

Therefore, the first frame F1 and the second frame F2 may be easily fastened only by a fastening operation of covering the cover 12 on the case 11, and at this time, the tube device 40 may be aligned at the correct position by using the alignment protrusions A and the alignment grooves H.

In addition, as shown in FIG. 3, in order to amplify the speckle pattern generated due to multiple scattering, a main exit optical axis or the light incident portion 31a of the laser source unit 31 may be formed to be inclined at a first angle K1 toward the camera unit 32 with respect to the vertical direction of the frame F so that a main optical path can be extended as long as possible in a zigzag fashion. Here, for example, the first angle K1 may be 0 degrees to 90 degrees.

Accordingly, as shown in FIG. 3, the laser light L emitted from the light incident portion 31a may exit through the light exit portion 32a in a state in which a speckle pattern generated due to multiple scattering of the laser light L that is scattered several times through the light scattering surface T and reflected in a zigzag fashion is maximized.

In this case, the light incident portion 31a and the light exit portion 32a may be formed to be spaced apart by a first distance L1 in the longitudinal direction of the frame F.

The first distance L1 may be optimized according to the intensity of the laser light L and the shape of the speckle pattern.

On the other hand, as shown in FIGS. 2 and 3, the device 200 for delivery of an infusate for humans according to some other embodiments of the present invention may further include an infusate flow blocking device 60 formed in the body 10 and configured to operate a knob N of a valve V installed in the tube device 40 or pressurize the tube device 40 in order to block the flow of the infusate 1 inside the tube device 40.

More specifically, for example, the infusate flow blocking device 60 may include a first valve knob-turning device 61 configured to turn a first knob N1 of a first valve V1 installed at a front end of the optical portion 40-2 of the tube device 40 and a second valve knob-turning device 62 configured to turn a second knob N2 of a second valve V2 installed at a rear end of the optical portion 40-2 of the tube device 40.

Accordingly, the contamination of the infusate 1 may be more precisely inspected in a state where the flow of the infusate 1 accommodated in the optical portion 40-2 is blocked.

At the same time, if the contamination of the infusate 1 is determined, this is warned of, and the injection of the contaminated infusate 1 into the human body or the living body of an animal or plant may be blocked in advance by using the infusate flow blocking device 60.

Therefore, it is possible to prevent medical incidents in advance by making it possible to determine contamination by bacteria or microorganisms in real-time or periodically.

On the other hand, as shown in FIGS. 2 and 3, the device 200 for delivery of an infusate for humans according to some other embodiments of the present invention may further include a pressure regulating device 70 formed in the body 10 and configured to regulate a pressure or flow rate inside the tube device 40.

Accordingly, the amount of the infusate 1 to be injected into the human body or the living body of an animal or plant may be controlled by precisely adjusting a fluid pressure or flow rate inside the tube device 40 by use of the pressure regulating device 70.

In addition, for example, as shown in FIGS. 2 and 3, the device 200 for delivery of an infusate for humans according to some other embodiments of the present invention may further include a control unit 80 configured to apply a control signal to the above-described pump unit 20, the infusate contamination sensing unit 30, the laser source unit 31, the camera unit 32, the valve V, the infusate flow blocking device 60, the first valve knob-turning device 61, the second valve knob-turning device 62, the pressure regulating device 70, and the like.

Accordingly, by using the control unit 80, it is determined in real-time or periodically whether the infusate 1 is contaminated, and when it is determined that the infusate 1 is not contaminated, the pump unit 20 may be controlled to normally inject the infusate 1 into the human body or the living body of an animal or plant, or when it is determined that the infusate 1 is contaminated, the injection of the infusate 1 may be stopped and a user may be warned of this for follow-up processes.

On the other hand, as shown in FIGS. 2 and 3, the tube device 40 according to some embodiments of the present invention, which is the tube device 40 applicable to the devices 100 and 200 for delivery of an infusate for humans of the present invention described above, may include a non-optical portion 40-1 made of a flexible material to correspond to the pump unit 20, the optical portion 40-2 made of a light-transmitting material to correspond to the infusate contamination sensing unit 30, and a valve V connected to the non-optical portion 40-1 at one end thereof, connected to the optical portion 40-2 at the other end thereof, and capable of blocking a flow of the infusate 1.

Here, as shown in FIGS. 2 and 3, the valve V includes the first valve V1 formed at the front end of the optical portion 40-2 and the second valve V2 formed at the rear end of the optical portion 40-2.

Therefore, the user may use the tube device 40 for one-time use by attaching it to the frame F, then separate the tube device 40 from the frame F and dispose of the tube device 40 easily, and thus it is possible to hygienically use the tube device 40.

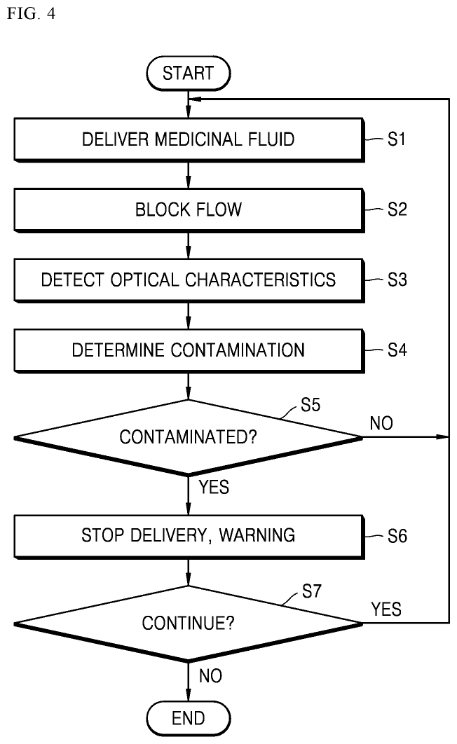

FIG. 4 is a flowchart illustrating a method of injecting an infusate for humans according to some embodiments of the present invention.

As shown in FIG. 4, a method of injecting an infusate for humans according to some embodiments of the present invention, which uses a device 100 or 200 for delivery of an infusate for humans that includes a body 10, a pump unit 20 installed in the body 10 and configured to form a pressure difference inside a tube device 40 so as to deliver an infusate for humans, and an infusate contamination sensing unit 30 installed in the body 10 and capable of sensing contamination of the infusate 1 by bacteria or microorganisms in a non-contact manner by using optical characteristics of the infusate 1, may include (S1) delivering the infusate 1 using the pump unit 20, (S2) blocking or controlling a flow of the infusate 1 using a valve V, (S3) detecting the optical characteristics of the infusate 1 using the infusate contamination sensing unit 30, (S4) determining contamination of the infusate 1 by bacteria or microorganisms by using the optical characteristics, (S5) when it is determined that the infusate is contaminated by bacteria or microorganisms, (S6) stopping delivery of the infusate 1 and warning of the same, and (S7) repeating the above-described series of processes in real-time or periodically by asking whether to continue the processes.

Meanwhile, the device and method for delivery of an infusate for humans of the present invention are not necessarily limited to the human body and may be applied to various animals or plants.

Although the present invention has been described in connection with the exemplary embodiment shown in the drawings, it is only illustrative. It will be understood to those skilled in the art that various modifications and equivalents can be made without departing from the scope and spirit of the invention. Therefore, the scope of the present invention should be defined only by the appended claims.

### INDUSTRIAL APPLICABILITY

According to an embodiment of the present invention as described above, it is possible to prevent medical incidents in advance by making it possible to determine in real-time or periodically contamination by bacteria or microorganisms before the human injection is injected into the human body or the living body of an animal or plant.

1. A device for delivery of an infusate for human, comprising:
a body;
a pump unit installed in the body and forming a pressure difference inside the tube device so as to deliver an infusate for humans; and
an infusate contamination sensing unit installed in the body and capable of sensing contamination of the infusate for humans by bacteria or microorganisms in a non-contact manner by using the optical characteristics of the infusate for humans.

2. The device of claim 1, wherein the infusate contamination sensing unit is a non-contact speckle sensor capable of emitting laser light to the infusate for humans and detecting a speckle pattern generated due to multiple scattering for the infusate.

3. The device of claim 2, wherein the infusate contamination sensing unit comprises
a frame formed in a shape surrounding at least a portion of the tube device;
a laser source unit formed on one side of the frame and configured to emit the laser light to the tube device through a light incident portion formed in the frame; and
a camera unit formed on the other side of the frame and capable of photographing a multiple-scattering speckle pattern received through a light exit portion formed in the frame.

4. The device of claim 3, wherein the frame has an inner diameter surface formed therein corresponding to the tube device and a light scattering surface or a reflective surface for scattering the laser light is formed on the inner diameter surface.

5. The device of claim 3, wherein the frame comprises
a first frame formed in a case of the body and having a first inner diameter surface formed therein corresponding to one side surface of the tube device and
a second frame having a second inner diameter surface formed therein corresponding to the other surface of the tube device and formed on a cover covering the case of the body so as to be fastened to the first frame.

6. The device of claim 5, wherein alignment grooves into which ring-shaped alignment protrusions of the tube device are inserted are formed on the first inner surface and the second inner surface.

7. The device of claim 5, wherein the frame further comprises a fastening device configured to fasten the first frame and the second frame.

8. The device of claim 7, wherein the fastening device is a magnetic object installed in the first frame or the second frame.

9. The device of claim 3, wherein a main exit optical axis or the light incident portion of the laser source unit is formed to be inclined at a first angle toward the camera unit with respect to a vertical direction of the frame.

10. The device of claim 3, wherein the light incident portion and the light exit portion are formed to be spaced apart by a first distance in a longitudinal direction of the frame.

11. The device of claim 1, further comprising an infusate flow blocking device formed in the body and configured to operate a knob of a valve installed in the tube device or pressurize the tube device in order to block a flow of the infusate inside the tube device.

12. The device of claim 11, wherein the infusate flow blocking device comprises a first valve knob-turning device configured to turn a first knob of a first valve installed at a front end of the optical portion of the tube device; and a second valve knob-turning device configured to turn a second knob of a second valve installed at a rear end of the optical portion of the tube device.

13. The device of claim 1, further comprising a pressure regulating device formed in the body and configured to regulate a pressure or flow rate inside the tube device.

14. The device of claim 1, wherein the tube device comprises a non-optical portion made of a flexible material to correspond to the pump unit and an optical portion made of a light-transmitting material to correspond to the infusate contamination sensing unit.

15. A method of injecting an infusate for humans, which uses a device for delivery of an infusate for humans that includes a body, a pump unit installed in the body and forming a pressure difference inside the tube device so as to deliver an infusate for humans, and an infusate contamination sensing unit installed in the body and capable of sensing contamination of the infusate for humans by bacteria or microorganisms in a non-contact manner by using the optical characteristics of the infusate for humans, the method comprising:
delivering the infusate for humans using the pump unit;
blocking or controlling a flow of the infusate for humans by using a valve;
detecting the optical characteristics of the infusate for humans by using the infusate contamination sensing unit;
determining contamination of the infusate for humans by bacteria or microorganisms by using the optical characteristics; and
when it is determined that the infusate is contaminated by bacteria or microorganisms, stopping delivery of the infusate for humans and warning of the same.

16. A tube device that is applicable to a device for delivery of an infusate for humans including a body, a pump unit installed in the body and forming a pressure difference inside the tube device so as to deliver an infusate for humans, and an infusate contamination sensing unit installed in the body and capable of sensing contamination of the infusate for humans by bacteria or microorganisms in a non-contact manner by using the optical characteristics of the infusate for humans, the tube device comprising:
a non-optical portion made of a flexible material to correspond to the pump unit;
an optical portion made of a light-transmitting material to correspond to the infusate contamination sensing unit; and
a valve connected to the non-optical portion at one end thereof, connected to the optical portion at the other end thereof, and capable of blocking a flow of the infusate for humans.

17. The tube device of claim 16, wherein the valve comprises a first valve formed at a front end of the optical portion and a second valve formed at a rear end of the optical portion.
